SEQUENCE LISTING (19 sequences)

SEQ ID NO: 1 - DNA, 46 bp, Artificial Sequence
  B CybC Fw NcoI primer
gggggccatggggcgtaaaagcctgttagctattcttgcagtctcc

SEQ ID NO: 2 - DNA, 43 bp, Artificial Sequence
  B CybC Rev Bam HI primer
gggggggatccttaacgatacttctggtgataggcgttgcggg

SEQ ID NO: 3 - DNA, 41 bp, Artificial Sequence
  CybC Fw w/o SP primer
gggggccatggccgctgatcctgaagacaatatggaaaccc

SEQ ID NO: 4 - DNA, 41 bp, Artificial Sequence
  K CybC Rev Bam HI primer
gggggggatccttaacgatacttcttgtgatatgaattgcg

SEQ ID NO: 5 - DNA, 390 bp, Escherichia coli
atggggcgtaaaagcctgttagctattcttgcagtctcctcgttggtattcagttctgcgtcgtttgccgctgatcttgaagacaatatggaaaccctcaacgacaatttaaaagtgatcgaaaaagcggataacgcggcgcaagtcaaagacgcgttaacgaagatgcgcgccgcagccctggatgcgcaaaaagcaacgccgccgaagctcgaagataaatcaccggacagcccggaaatgaaagatttccgccacggtttcgacattctggtcggtcagattgacgacgcgctgaagctggcaaatgaaggtaaagtaaaagaagcgcaggctgctgcagagcaactgaaaacgacccgcaacgcctatcaccagaagtatcgttaa

SEQ ID NO: 6 - protein, 129 aa, Escherichia coli
MGRKSLLAILAVSSLVFSSASFAADLEDNMETLNDNLKVIEKADNAAQVKDALTKMRAAALDAQKATPPKLEDKSPDSPEMKDFRHGFDILVGQIDDALKLANEGKVKEAQAAAEQLKTTRNAYHQKYR

SEQ ID NO: 7 - DNA, 390 bp, Escherichia coli
atggggcgtaaaagcctgttagctattcttgcagtctcctcgttggtattcagttctgcgtcgtttgctgctgatctcgaagacaatatggaaaccctcaacgacaatttaaaagtggtcgaaaaagccgataacgcggcgcaagtcaaagacgcgttaacgaagatgcgcgccgcagcgctggatgcgcaaaaagcaacgccgccgaagctcgaagataaatcaccggacagcccggaaatgaaagatttccgccacggtttcgacattctggtcggtcagattgacgacgcgctgaagctggcaaatgaaggtaaagtaaaagaagcgcaggctgctgcagagcaactgaaaacgacccgcaattcatatcacaagaagtatcgttaa

SEQ ID NO: 8 - protein, 129 aa, Escherichia coli
MGRKSLLAILAVSSLVFSSASFAADLEDNMETLNDNLKVVEKADNAAQVKDALTKMRAAALDAQKATPPKLEDKSPDSPEMKDFRHGFDILVGQIDDALKLANEGKVKEAQAAAEQLKTTRNSYHKKYR

SEQ ID NO: 9 - protein, 110 aa, E. coli B cybC
MRKSLLAILAVSSLVFSSASFAADLEDNMETLNDNLDNAAQVKDALTKMRAAALDAQKATPPKLEDKSPDSPEGFDILVGQIDDALKLANEGKVKEAQAAAEQLKTTRNA

SEQ ID NO: 10 - protein, 103 aa, E. coli K12
ILAVSSLVFSSASFAADLEDNMETLNDNLDNAAQVKDALTKMRAAALDAQKATPPKLEDKSPDSPEGFDILVGQIDDALKLANEGKVKEAQAAAEQLKTTRNS

SEQ ID NO: 11 - protein, 82 aa, E. coli 0157 H7
METLNDNLDNAAQVKDALTKMRAAALDAQKATPPKLEDKSPDSPEGFDILVGQIDDALKLANEGKVKEAQAAAEQLKTTRNS

SEQ ID NO: 12 - protein, 110 aa, S. typhi
MRKSLLAILAVSSLVFGSAVFAADLEDNMDILNDNLDSAPELKAALTKMRAAALDAQKATPPKLEDKAPDSPEGFDILVGQIDGALKLANEGNVKEAKAAAEALKTTRNT

SEQ ID NO: 13 - protein, 111 aa, K. pneu Kcyb
MGRKKRLAMLAVSAFALGSASAFADLGEDMDTLAENLSDAGELKAALNKMRTAAVDAQKETPPKLEGKAADSAEGLDILIGQIDGALKLANEGKVKEAQAAAEEFKTTRNT

SEQ ID NO: 14 - protein, 110 aa, Y. pestis
MGKTLMALITAALLSTSSLVMAASVADDMETIAEHYDSTAVIKQDLQAMRVAAVDAQKGIPTKLKSKVEDSPEGMDVLIGEIDGALALADQGKLDEAKQAAQDFKDTRNT

SEQ ID NO: 15 - protein, 106 aa, P. multocida
MHKLLKLLSITLIGLSVATGVQANVRAEMNQMKTVAKDVAEFQESAKILREIAQQSSEKRPSSITNDADGMKEFITALDEADKLAQEGNLDAAKTAAKKLFDIRNV

SEQ ID NO: 16 - DNA, 420 bp, E. coli BcybC
atgcgtaaaagcctgttagctattcttgcagtctcctcgttggtattcagttctgcgtcgtttgccgctgatcttgaagacaatatggaaaccctcaacgacaatttaaaagtgatcgaaaaagcggataacgcggcgcaagtcaaagacgcgttaacgaagatgcgcgccgcagcgctggatgcgcaaaaagcaacgccgccgaagctcgaagataaatcaccggacagcccggaaatgaaagatttccgccacggtttcgacattctggtcggtcagattgacgacgcgctgaagctggcaaatgaaggtaaagtaaaagaagcgcaggctgctgcagagcaactgaaaacgacccgcaacgcctataccagaagtatcgttaattcctcatttccctgttgcctgcactcaggtaac

SEQ ID NO: 17 - protein, 128 aa, E. coli BcybC [sequence omitted]

SEQ ID NO: 18 - DNA, 401 bp, K. pneu Kcyb
atggggcgaaaaaaacggttagcgatgctggctgtctctgcttttgcgctcggttcagcgtcggccttcgccgacctgggcgaagacatggacactctggcagaaaacctgcaagtggtacagaagacctccgacgccggcgagctgaaagcggcgctgaataagatgcgtaccgccgcggtcgatgctcagaaagagaccccgccgaagctggaaggcaaagcggccgacagcgctgagatgaaagattaccgtcacggtctggatattctgatcggccagatcgacggtgcgctgaagctggcgaacgaaggcaaggtgaaagaggcgcaggccgccgcggaggagtttaaaaccacccgcaacacctatcataagaagtaccgctaaccgttcttttc

SEQ ID NO: 19 - protein, 129 aa, K. pneu Kcyb
MGRKKRLAMLAVSAFALGSASAFADLGEDMDTLAENLQVVQKTSDAGELKAALNKMRTAAVDAQKETPPKLEGKAADSAEMKDYRHGLDILIGQIDGALKLANEGKVKEAQAAAEEFKTTRNTYHKKYR

(19) United States
(12) Patent Application Publication
(10) Pub. No.: US 2005/0067278 A1
(43) Pub. Date: Mar. 31, 2005

(54) Oxygen electrode

(76) Inventor: Koji Sode, Tokyo (JP)

Correspondence Address: BIRCH STEWART KOLASCH & BIRCH, PO BOX 747, FALLS CHURCH, VA, 22040-0747, US

(21) Appl. No.: 10/471,624

(22) PCT Filed: Mar. 8, 2002
(86) PCT No.: PCT/JP02/02191

(30) Foreign Application Priority Data
Mar. 13, 2001 (JP) 2001-070421
Sep. 17, 2001 (JP) 2001-281985

(51) Int. Cl. C12M 1/00; G01N 1/00

(52) U.S. Cl. 204/403.04; 204/403.1

(57) ABSTRACT

Disclosed is an enzyme electrode having an oxidoreductase (for instance, glucose oxidase, cholesterol oxidase, fructosylamine oxidase and glucose dehydrogenase) and an electron-transfer protein (for instance, cytochrome C, cytochrome b562 and cytochrome c551), as well as a sensor utilizing the enzyme electrode as working electrode. The enzyme electrode of the invention can provide high response current values.

### TECHNICAL FIELD

The present invention relates to an enzyme electrode and a biosensor that uses the enzyme electrode.

### Technical Background

An enzyme electrode is an electrode in which an enzyme is immobilized on the surface of an electrode such as a gold electrode, platinum electrode or carbon electrode. Enzyme electrodes are broadly used as biosensors that exploit the reaction specificity of an enzyme to detect specifically a variety of biologically active substances.

For instance, glucose sensors that measure simply and rapidly the blood glucose level have been developed. As glucose sensor element, glucose oxidase (GOD) is mostly used. Because GOD is an enzyme that is heat-stable and can be supplied inexpensively in large amounts, it has been used frequently. Furthermore, addition of a variety of electron mediators such as potassium ferricyanide to the measurement system has been attempted in order to decrease the voltage applied to the electrode to lower the influence of contaminant substances. In addition, it is possible to use glucose dehydrogenase (GDH) as a mediator type sensor element that is unaffected by the concentration of dissolved oxygen. For instance, the use of co-enzyme-linked type PQQ glucose dehydrogenase (PQQGDH) has been disclosed (JP A 10-243786, WO00/66744, WO00/61730).

In addition, enzyme electrodes for measuring the concentrations of cholesterol and fructosylamine in blood have been studied using cholesterol oxidase and fructosylamine oxidase (Electrochemistry, 68 (11), 869-871, 2000).

However, when these oxidoreductases were applied to enzyme electrodes, there was the problem that the response currents from the electrodes were low. This is due to the fact that the electron transfer from these oxidoreductases to the electrode or the electron mediator is slow.

Consequently, the object of the present invention is to provide an enzyme electrode with which a high response current value can be obtained.

### DISCLOSURE OF THE INVENTION

It has now been discovered that enzyme electrodes having a high response value could be obtained by immobilizing an electron-transfer protein together with an oxidoreductase on the electrode. Thus, the present invention provides an enzyme electrode that possesses an oxidoreductase and an electron-transfer protein thereon.

An oxidoreductase designates an enzyme that catalyzes oxidization-reduction reaction. Preferably, the oxidoreductase is an oxidoreductase having pyrroloquinoline quinone as coenzyme, or an oxidoreductase having flavin as coenzyme. More preferably, the oxidoreductase is selected from the group consisting of glucose oxidase, cholesterol oxidase, lactate oxidase, alcohol oxidase, galactose oxidase, bilirubin oxidase, fructosylamine oxidase, glucose dehydrogenase, alcohol dehydrogenase and glucose-3-dehydrogenase.

An electron-transfer protein designates a protein that can, in a biological oxido-reduction system, receive an electron from an electron donor and become reduced, then donate an electron to an electron acceptor and become oxidized. Preferably, electron-transfer proteins are cytochrome b and cytochrome C, more preferably, cytochrome b562. Preferably, the cytochrome b562 used as the electron-transfer protein is the cytochrome b562 derived from Escherichia coli. In addition, as the cytochrome b562 used as the electron-transfer protein, cytochrome b562 derived from Acinetobacter calcoaceticus, Klebsiella pneumoniae or other bacteria may be exploited. More preferably, the cytochrome b562 is a recombinant protein produced in Escherichia coli.

By immobilizing an electron-transfer protein together with an oxidoreductase on an electrode, electron-transfer from the oxidoreductase to the electrode or to the electron mediator can be accelerated, thereby making it possible to obtain an enzyme electrode having a high response current value. As an embodiment of the present invention, the electron-transfer system containing PQQ glucose dehydrogenase, cytochrome b562 and an electron mediator is shown in FIG. 1.

Particularly preferably, the enzyme electrode of the present invention is selected from the combinations of oxidoreductase and electron-transfer protein below: glucose oxidase and cytochrome b562, cholesterol oxidase and cytochrome b562, lactate oxidase and cytochrome b562, fructosylamine oxidase and cytochrome b562, glucose dehydrogenase and cytochrome b562, glucose dehydrogenase that has pyrroloquinoline quinone as the coenzyme (PQQGDH) and cytochrome b562, glucose dehydrogenase that has flavin as the coenzyme and cytochrome b562.

It is possible to manufacture the enzyme electrode of the present invention by immobilizing these oxidoreductases and electron-transfer proteins on the surface of the electrode. Preferably, these oxidoreductases and electron-transfer proteins are attached to the electrode in a state so as to be chemically cross-linked. Cross-linking can be carried out for instance with glutaraldehyde.

In addition, in a particularly preferable embodiment of the present invention, the enzyme electrode of the present invention can provide a high response current value, even in such systems that do not contain an electron mediator.

In another aspect, the present invention provides a sensor characterized in that it utilizes the above-mentioned enzyme electrode of the present invention as a working electrode.

When used in the present specification, a sensor designates a measurement system that measures electrochemically the concentration of an analyte, and in general contains three electrodes: a working electrode (enzyme electrode), a counter electrode (platinum and the like) and a reference electrode (Ag/AgCl). Alternatively, this may be a two-electrode system constituted of a working electrode and a counter electrode, which is commonly used in conventional simple blood glucose level systems. The sensor may further contain a constant temperature cell that holds the buffering solution and the analyte sample, a power source to apply voltage to the working electrode, an ampere meter, and a recorder. The sensor may be of a batch type or flow type. Such an enzyme sensor structure is well known in the art, and is mentioned for instance in Biosensors—Fundamental and Applications—Anthony P. F. Tuner, Isao Karube and George S. Wilson, Oxford University Press 1987.

Preferably, the sensor of the present invention further contains an electron mediator. An electron mediator designates an oxido-reductive substance such as non-proteic metal complexes and organic compounds, which mediates electron-transfer from the oxidoreductase to the electrode. Electron mediators include, for instance, potassium ferricyanide, phenazine methosulfate, ferrocene and derivatives thereof.

### BRIEF DESCRIPTION OF THE DRAWINGS

FIG. 1 shows electron-transfer in a system that uses PQQ glucose dehydrogenase, cytochrome b562 and an electron mediator.

FIG. 2 shows responses to glucose sample injection of a sensor (A) in which PQQ glucose dehydrogenase and cytochrome C are immobilized by cross-linking and which has potassium ferricyanide as an electron mediator, and a sensor (B) in which PQQ glucose dehydrogenase is immobilized alone and which has potassium ferricyanide as an electron mediator.

FIG. 3 shows glucose concentration dependency of the value of the response current to glucose sample injection for sensor (A) in which PQQ glucose dehydrogenase and cytochrome C are immobilized by cross-linking and which has potassium ferricyanide as an electron mediator, and sensor (B) in which PQQ glucose dehydrogenase is immobilized alone and which has potassium ferricyanide as electron mediator.

FIG. 4 shows glucose concentration dependency of the value of the response current to glucose sample injection for a sensor in which PQQ glucose dehydrogenase and cytochrome b562 are immobilized by cross-linking and which has potassium ferricyanide as an electron mediator, and a sensor in which PQQ glucose dehydrogenase is immobilized alone and which has potassium ferricyanide as an electron mediator. In the figure, “GB25U+100cyt b-562” designates an electrode in which 100 times molar excess of cytb562 to 25U of PQQGDH us immobilized, “GB25U+1cyt b-562” designates an electrode in which equal molar of cytb562 to 25U of PQQGDH is immobilized, “100cyt b-562 only” designates an electrode in which an amount of cytb562 equal to that in the above-mentioned GB25U+100cyt b-562 is immobilized, and “GB25U only” designates an electrode in which only 25U of PQQGDH is immobilized.

FIG. 5 shows glucose concentration dependency of the value of the response current to glucose sample injection for a sensor that was created by immobilizing PQQ glucose dehydrogenase and cytochrome b562, without adding an electron mediator. In the figure, “GB25U+100cyt b-562” designates an electrode in which 100 times molar excess of cytb562 to 25U of PQQGDH is immobilized, and “GB25U+1cyt b-562” designates an electrode in which equal molar of cytb562 to 25U of PQQGDH is immobilized.

FIG. 6 shows glucose concentration dependency of the value of the response current to glucose sample injection for a sensor in which glucose oxidase and cytochrome b562 are immobilized by cross-linking and which uses potassium ferricyanide as electron mediator.

FIG. 7 shows glucose concentration dependency of the value of the response current to glucose sample injection for a sensor that was created by immobilizing glucose oxidase and cytochrome b562, without adding an electron mediator.

FIG. 8 shows cholesterol concentration dependency of the value of the response current to cholesterol sample injection for a sensor in which cholesterol oxidase and cytochrome b562 are immobilized by cross-linking and which uses potassium ferricyanide as electron mediator.

FIG. 9 shows fructosyl-valine concentration dependency of the value of the response current to fructosyl-valine sample injection for a sensor that was created by immobilizing fructosylamine oxidase and cytochrome b562, without adding an electron mediator.

FIG. 10 shows identity and similarity of sequences of cyt.b562 from a variety of facultative anaerobic enteric bacteria genomes that show homology with E. coli B-derived cyt.b562.

FIG. 11 shows regions of amino acid sequence of a variety of facultative anaerobic enteric bacteria genomes that show homology to E. coli B-derived cyt.b562.

FIG. 12 shows the amino acid sequences of E. coli B-derived cyt.b562 and K. pneumoniae-derived cytb562, and the sequences of the genes that code these cytochromes. The amino acid residues that are enclosed in frames are conserved amino acids that coordinate the haeme iron.

FIG. 13 shows the reduction of K. pneumoniae-derived cytochrome b562, upon addition of glucose in the presence of PQQGDH.

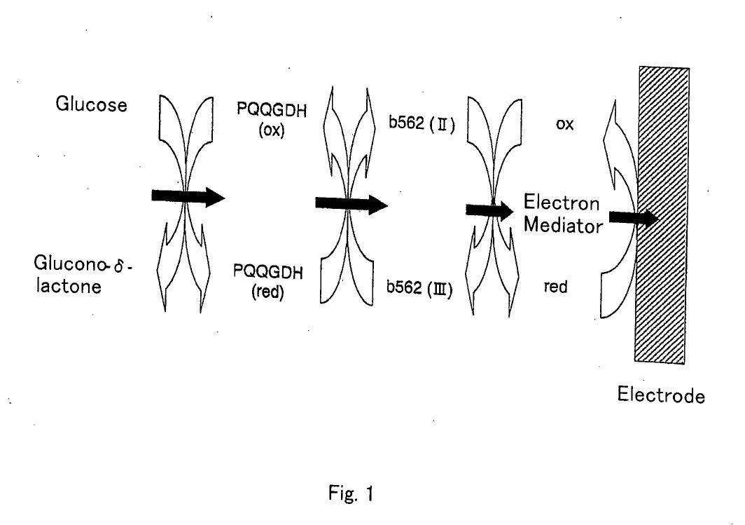

### PREFERRED EMBODIMENT

The enzyme electrode of the present invention is characterized in that an oxidoreductase and an electron-transfer protein are immobilized on its surface. The enzyme electrode of the present invention exhibits a higher responsiveness than enzyme electrodes that have oxidoreductase alone immobilized.

Examples of oxidoreductase that may be used in the present invention include glucose oxidase, cholesterol oxidase, lactate oxidase, alcohol oxidase, galactose oxidase, bilirubin oxidase, fructosylamine oxidase, glucose dehydrogenase, alcohol dehydrogenase and glucose-3-dehydrogenase. Glucose dehydrogenase, which uses pyrroloquinoline quinone as coenzyme (abbreviated as “PQQGDH” in the present specification) is particularly preferred. PQQGDH is an enzyme that catalyzes a reaction whereby glucose is oxidized to generate gluconolactone, and can be used as an element of a glucose sensor. The presence of PQQGDH has been demonstrated in several strains of Acinetobacter calcoaceticus (Biosci. Biotech. Biochem. (1995), 59 (8), 1548-1555), its structural gene cloned and amino acid sequence elucidated (Mol. Gen. Genet. (1989), 217:430-436).

Preferably, water-soluble PQQGDH, particularly preferably water soluble PQQGDH derived from Acinetobacter calcoaceticus, is used in the enzyme electrode of the present invention. Water-soluble PQQGDH can be isolated form the same bacteria, or recombinantly produced in Escherichia coli as shown in Koji Sode, et al., Enz. Microbiol. Technol., 26, 491-496 (2000). Alternatively, water-soluble PQQGDH may be a modified PQQGDH with increased heat resistance as shown in WO00/61730, or a modified PQQGDH with increased substrate specificity as shown in WO00/66744.

The oxidoreductase used in the present invention may be a modified oxidoreductase, resulting from the chemical modification of part of the structure of a natural oxidoreductase. Such a modified enzyme can be made, for instance, by substituting one or more amino acid residues of the enzyme protein with another natural or non-naturally existing amino acid residue, or by deleting or adding one or more amino acid residues.

Examples of electron-transfer protein used in the present invention include cytochrome C. There is no particular restriction as far as the origin of cytochrome C is concerned, and for example, horse heart-derived cytochrome C sold by Sigma may be used. In addition, cytochrome b562 may also be used as the electron-transfer protein. There is no restriction as far as the origin of cytochrome b562 is concerned, and, for example, Escherichia coli-derived cytochrome b562 may be used. Escherichia coli-derived cytochrome b562 may be prepared by culturing Escherichia coli and purifying the protein from the cell lysate. A method for preparing cytochrome b562 from Escherichia coli is described, for instance, in E. Itagaki and L. P. Hager, Biochem. Biophys. Res. Commun., 32, 1012-1019 (1968), F. Lederer et al., J. Mol. Biol., 148, 427-448 (1981). In addition, since Escherichia coli-derived cytochrome b562 is a protein that is secreted in the periplasm, it may be prepared by destroying the extra-cellular membrane by a method such as osmotic shock, and purifying cytochrome b562.

Alternatively, cytochrome b562 may be prepared by isolating the structural gene of Escherichia coli B strain-derived cytochrome b562 from the Escherichia coli genome, inserting it into an expression vector that functions in Escherichia coli such as pTrc99A, to construct recombinant Escherichia coli, then culturing the recombinant Escherichia coli, and purifying cytochrome b562 from the cell lysate thereof. The gene sequence of Escherichia coli B strain-derived cytochrome b562 is described in Eur. J. Biochem. 202 (2), 309-313 (1991).

In addition, a gene similar to Escherichia coli B strain-derived cytochrome b562 has been cloned from E. coli K strain (Tower, M. K., Biochem. Biophys. Acta. 1143, 109-111 (1993)). This gene is inactive, and compared to the B strain-derived cytochrome b562, seven residues at the N-terminus are missing, and mutations exist at three loci in the cytochrome b562 protein (Ile40Val, Ala123Ser and Gln126Lys, where Met at the N-terminus of the B strain-derived b562 is represented by the 1-position). The region of the E. coli K strain-derived cytochrome b562 gene that codes for the mature protein (from Ala24 to Arg129 of SEQ ID NO: 6) may be inserted into a secretion expression vector that functions in Escherichia coli to construct a recombinant Escherichia coli, and cytochrome b562 may be prepared from this Escherichia coli. Also, the region of the E. coli B strain-derived cytochrome b562 gene that codes for the mature protein (from Ala24 to Arg129 of SEQ ID NO: 8) may be inserted into a secretion expression vector that functions in Escherichia coli to construct a recombinant Escherichia coli, and cytochrome b562 may be prepared from this Escherichia coli. Alternatively, a portion of the B strain-derived cytochrome b562 gene and a portion of the K strain-derived cytochrome b562 gene may be ligated, and inserted in an expression vector that functions in Escherichia coli such as pTrc99A to construct a recombinant Escherichia coli, then the recombinant Escherichia coli may be cultured and chimeric cytochrome b562 may be prepared from the cell lysate thereof. In addition, cytochrome b562 derived from bacteria such as S. typhi, S. typhinulium, K. pneumomiae, Y. pestis, P. multocida and S. pneumoniae may also be used.

Further, electron-transfer protein used in the present invention may be a modified electron-transfer protein, resulting from the chemical modification of part of the structure of a natural protein. Such a modified protein can be made, for instance, by substituting one or more amino acid residues of the protein with another natural or non-naturally existing amino acid residue, or by deleting or adding one or more amino acid residues.

A carbon electrode, gold electrode or platinum electrode may be used as an electrode used in the enzyme electrode of the present invention. A carbon paste electrode is particularly preferable.

To manufacture the enzyme electrode of the present invention, an oxidoreductases and an electron-transfer protein are mixed to prepare a protein mixture. The protein mixture recognizes the presence of the analyte (for instance glucose) on the enzyme electrode, catalyzes oxido-reduction reaction, and transmits the electrons generated by the reaction to the electrode. The mixing ratio of the oxidoreductase and the electron-transfer protein is generally 1:1 to 1:10000 by molar ratio, preferably 1:10 to 1:5000, and more preferably 1:50 to 1:1000. The protein mixture may be directly mixed with an electrode material, such as carbon paste, and attached to an electrode. Alternatively, immobilized enzyme may be prepared using a general enzyme immobilization method and attached onto an electrode. For instance, the protein mixture may be prepared by mixing the proteins and cross linking them with a bifunctional cross linking reagent, such as glutaraldehyde, or by entrapping them in synthetic polymers, such as photo-cross linking polymer, electric conductive polymer and oxido-reduction polymer, or natural macromolecular matrices. The protein mixture may be mixed with carbon paste or further cross linking after mixing with carbon paste, and attached onto an electrode made of carbon, gold or platinum.

It is also possible to immobilize an electron mediator together with the protein mixture onto an electrode. Typically, PQQGDH is mixed with cytochrome C or cytochrome b562, and is further mixed with carbon paste and then lyophilized. This is attached onto a carbon electrode, and immersed into a glutaraldehyde aqueous solution to crosslink the complex proteins, and used to fabricate the enzyme electrode.

The sensor of the present invention is characterized in that it has the above-mentioned enzyme electrode as the working electrode. For instance, a platinum electrode may be used as a counter electrode, and an Ag/AgCl electrode may be used as a reference electrode. The sensor of the present invention may further contain an electron mediator. Examples of the electron mediator include, but not limited to, potassium ferricyanide, phenazine methosulfate, ferrocene and derivatives thereof. Preferably, potassium ferricyanide is used.

Measurements of the concentration of the analyte, for instance glucose, may be carried out in the following way described below. Buffer solution is introduced in a constant temperature cell, and electron mediator is added and maintained at a constant temperature. Potassium ferricyanide or phenazine methosulfate may be used as mediator. An enzyme electrode on which PQQGDH and cytochrome C or cytochrome b562 have been immobilized is used as a working electrode, in combination with a counter electrode (for instance platinum electrode) and a reference electrode (for instance Ag/AgCl electrode). A constant voltage is applied to the working electrode, and after the current has stabilized, a glucose-containing sample is added into the constant temperature cell and the increase in current is measured. According to a calibration curve made from standard concentrations of glucose solution, the concentration of glucose in the sample can be calculated.

The contents of all the patents and references explicitly cited in the present invention are incorporated by reference in their entirety. Also the contents described in the specification of Japanese Patent Application Nos. 2001-70421 and 2001-281985, to which the present application claims priority, is incorporated herein by reference in their entirety.

### EXAMPLES

In the following, the present invention will be explained in detail by Examples, which do not limit the scope of the present invention.

### Example 1

### Preparation of Recombinant Cytochrome b562

According to Nikkila, H., Gennis, R. B. and Sligar, S. G., Eur. J. Biochem. 202 (2), 309-313 (1991) and Tower, M. K., Biochem. Biophys. Acta. 1143, 109-111 (1993), the following two sets of oligonucleotide primers were synthesized, and each was used for genomes of Escherichia coli, i.e., Escherichia coli DH5a strain (E. coli K strain) and Escherichia coli B strain to amplify the structural region of cytochrome b562 by the PCR method. The genomic DNA was extracted from respective Escherichia coli cells using a conventional method. As PCR primers, a primer that contains a sequence recognized by the restriction endonuclease Nco I and a sequence that amplifies and adds a region of E. coli B strain-derived signal sequence for secreting cytochrome b562 (B CybC Fw NcoI), and a primer that does not contain the signal sequence (CybC Fw w/o SP) were designed for the forward primers, and primers that contain a sequence recognized by the restriction endonuclease Bam HI (B CybC Rev Bam HI, K Cyb Rev Bam HI) were designed for the reverse primers.

(SEQ ID No: 1)

B CybC Fw NcoI;
5′-GGGGGCCATGGGGCGTAAAAGCCTGTTAGCTATTCTTGCAGTCTCC-
3′

(SEQ ID No: 2)

B CybC Rev Bam HI;
5′-GGGGGGGATCCTTAACGATACTTCTGGTGATAGGCGTTGCGGG-3′

(SEQ ID No: 3)

CybC Fw w/o SP;
5′-GGGGGCCATGGCCGCTGATCCTGAAGACAATATGGAAACCC-3′

(SEQ ID No: 4)

K CybC Rev BaM HI;
5′-GGGGGGGATCCTTAACGATACTTCTTGTGATATGAATTGCG-3′

PCR was performed with E. coli DH5a strain using the primer combination <B CybC Fw NcoI−K Cyb Rev Bam HI>or <CybC Fw w/o SP−K CybC Rev Bam HI>, and with E. coli B strain using the primer combination <B CybC Fw NcoI− B CybC Rev Bam HI>or <CybC Fw w/o SP− B CybC Rev Bam HI>, to amplify the respective regions.

Each of the amplified gene fragments was inserted into the NcoI-Bam HI site of the Escherichia coli expression vector Trc99A to build pTrc99A-KcybC and pTrc99A-KcybC w/o SP as well as pTrc99A-BcybC and pTrc99A-BcybC w/o SP respectively as vectors for the expression of cytochrome b562. These vectors were transformed into E. coli DH5a strain to create recombinant Escherichia coli capable of producing cytochrome b562.

The structural gene sequence and amino acid sequence of cytochrome b562 of E. coli B strain used in cloning and expression are shown in SEQ ID NOs: 5 and 6, respectively. The structural gene sequence and amino acid sequence of cytochrome b562 of E. coli K strain used in cloning and expression are shown in SEQ ID NOs: 7 and 8, respectively.

Recombinant Escherichia coli created in this way was cultured with shaking at 37° C. in L broth that contained 50 μg/ml of ampicillin. After collecting the bacterial cells, a cellular extract was obtained by sonication. Red color derived from cytochrome b562 was found in all culture of the recombinant Escherichia coli transformed with the expression vector, showing that cytochrome b562 was produced as a water soluble protein.

Among these, transformants with high productivity were the strains transformed with pTrc99A-KcybC and with pTrc99A-BcybC as expression vectors, in which cytochrome b562 gene containing the signal sequence was inserted. Both vectors were expressed in Escherichia coli, and E. coli K strain- or B strain-derived cytochrome b562 was produced in large amounts in the periplasm. Cytochrome b562 to be used in the construction of enzyme electrode was prepared using these recombinant Escherichia coli.

E. coli DH5a strain transformed with pTrc99A-BcybC or pTrc99A-KcybC was cultured in a fermenter at 37° C. in 2 liters of L broth containing 50 μg/ml of ampicillin. When the logarithmic growth phase was reached, 300 μM IPTG was added to induce expression of the recombinant gene, and cultivation was continued until the stationary phase was reached. The bacterial cells were collected and disrupted with a sonicator to obtain cellular extract, which was desalted by dialysis against a 10 mM MOPS pH7.2 buffer solution, and then purified by anion exchange chromatography with DEAE-Toyopearl. The molecular weight of the obtained protein was shown to be 12.3 kDa by SDS-PAGE. From the spectral analyses, a reduced spectrum at 562 nm, which is characteristic of cytochrome b562, was observed, indicating that a purified cytochrome b562 was prepared.

### Example 2

### Construction of an Enzyme Electrode in Which PQQGDH and Cytochrome C Have Been Immobilized

To an enzyme solution (3900 U/mg protein) of Acinetobacter calcoaceticus-derived water-soluble PQQGDH purified according to conventional method, was added 1 μM PQQ and 1 mM CaCl₂ at a final concentration, and incubated for 30 minutes at room temperature under dark conditions. The enzyme solution was dialyzed overnight against 100 volumes of a 10 mM MOPS buffer solution (pH7.0) containing 1 mM CaCl₂. Horse heart-derived cytochrome C (hereafter may be indicated by cyt.c) purchased from Sigma (No. C-7752) was dissolved in 10 mM MOPS buffer solution (pH7.0) at a final concentration of 1 mM, and was dialyzed overnight against 100 volumes of a 10 mM MOPS buffer solution (pH7.0).

PQQGDH (25 units, 0.64×10⁻¹⁰ mol) and cyt.c sample (100 times molar excess to the enzyme, i.e. 0.64×10⁻⁸ mol) prepared in this way were mixed together with 20 mg of carbon paste and lyophilized. After thorough mixing, the mixture was applied only to the surface of a carbon paste electrode which was already filled with approximately 40 mg of carbon paste, and polished on a filter paper.

This electrode (enzyme electrode) was treated in a 10 mM MOPS buffer solution (pH7.0) containing 1% glutaraldehyde for 30 minutes at room temperature, and further treated in a 10 mM Tris buffer solution (pH7.0) for 20 minutes at room temperature. This electrode was equilibrated in a 10 mM MOPS buffer solution (pH7.0) for one hour or more at room temperature.

### Example 3

### Measurement of Glucose Using a Sensor Constructed from PQQGDH, Cytochrome C (cyt.c) and Potassium Ferricyanide as an Electron Mediator

A 10 mM MOPS buffer solution (pH7.0) containing 1 mM CaCl₂ was placed in a constant temperature cell, potassium ferricyanide was added as a mediator at a final concentration of 10 mM, and the total volume was made to be 10 ml. The carbon paste electrode (enzyme electrode) constructed in Example 2, in which PQQGDH and cytochrome C are immobilized, as the working electrode, a platinum electrode as the counter electrode and an Ag/AgCl electrode as the reference electrode were inserted therein to construct the sensor.

Measurements were all performed at 25° C. An electric potential of +400 mV vs Ag/AgCl was applied. When the current became stationary, the current value that increased with the addition of different concentrations of glucose was measured. The current value when glucose was not added was defined as 0 A.

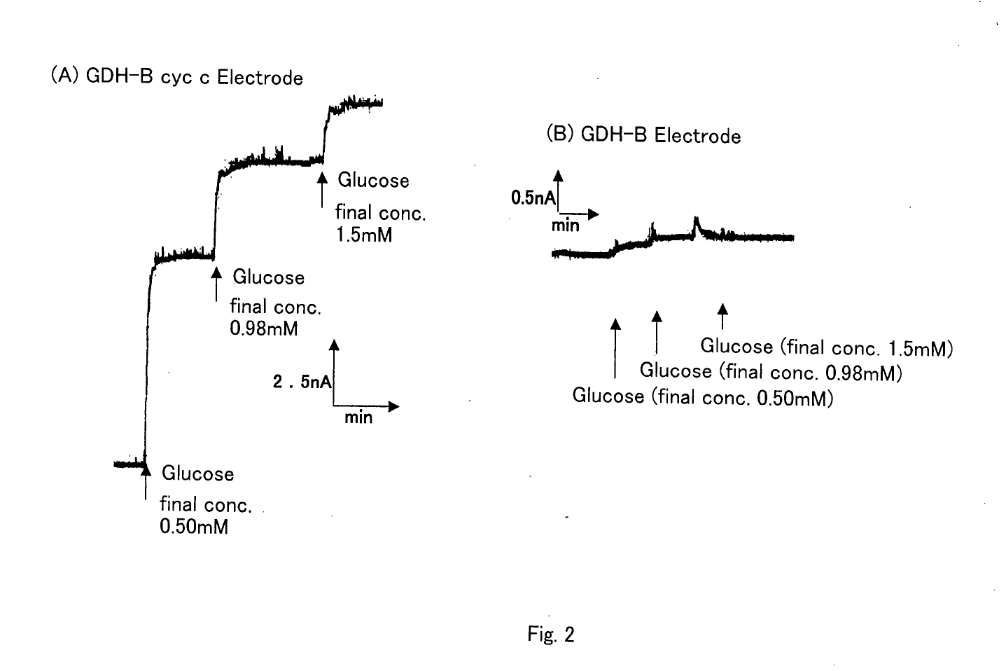

The enzyme electrode in which PQQGDH alone is immobilized and the enzyme electrode in which PQQGDH and 100 times molar excess of cyt.c is immobilized were used. The response to the injection of glucose sample in the presence of potassium ferricyanide as the electron mediator is shown in FIG. 2. The enzyme electrode in which PQQGDH and 100 times molar excess of cyt.c is immobilized showed a considerably higher response, demonstrating that it has a higher sensitivity.

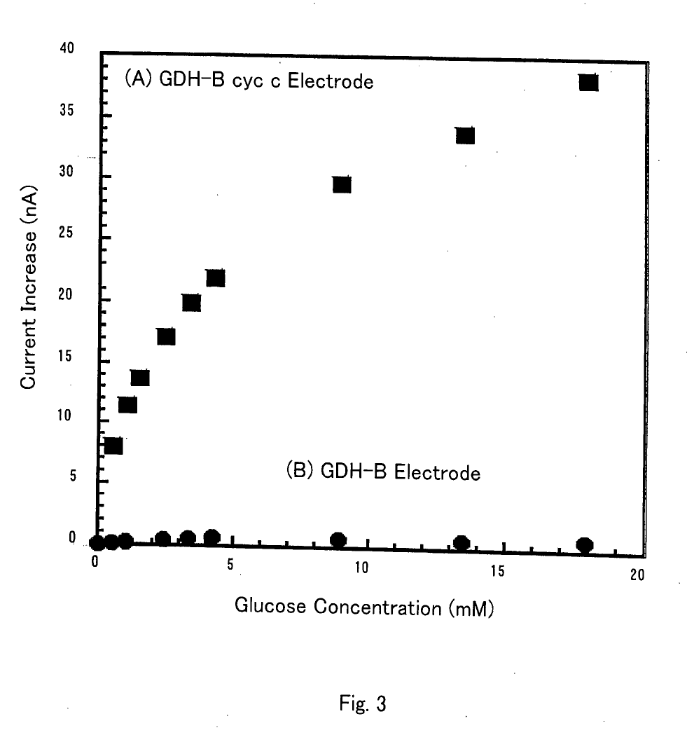

FIG. 3 shows the calibration curve of the enzyme electrode in which PQQGDH alone is immobilized and the enzyme electrode in which PQQGDH and 100 times molar excess of cyt.c are immobilized, in the presence of potassium ferricyanide as the electron mediator. The response current values of respective electrode at a glucose concentration of 4.2 mM were compared. The response current values of each electrode at a glucose concentration of 4.2 mM were 0.5 nA for the enzyme electrode in which PQQGDH alone is immobilized, and 22 nA for the enzyme electrode in which PQQGDH and 100 times molar excess of cyt.c is immobilized. The current value of the electrode with immobilized cyt.c in response to glucose was approximately 40 times higher compared to the electrode with the enzyme alone.

In addition, an increase in the response current value was observed when the amount of cyt.c immobilized onto the electrode was increased. The response current value was almost proportional to the quantity of cyt.c immobilized (data not shown).

### Example 4

### Construction of an Enzyme Electrode in Which PQQGDH and Cytochrome b562 are Immobilized

To an enzyme solution (3900 U/mg protein) of Acinetobacter calcoaceticus-derived water-soluble PQQGDH purified according to conventional method, was added 1 μM PQQ and 1 mM CaCl₂ at a final concentration, and incubated for 30 minutes at room temperature under dark conditions. The enzyme solution was dialyzed overnight against 100 volumes of a 10 mM MOPS buffer solution (pH7.0) containing 1 mM CaCl₂. Cytochrome b562 (cyt.b562) prepared as shown in Example 1 was dissolved in a 10 mM MOPS buffer solution (pH7.0) at a final concentration of 1 mM, and was dialyzed overnight against 100 volumes of a 10 mM MOPS buffer solution (pH7.0).

PQQGDH (25 units, 0.64×10⁻¹⁰ mol) and cyt.b562 sample (100 times molar excess to the enzyme, i.e., 0.64×10⁻⁸ mol) prepared in this way were mixed together with 20 mg of carbon paste and lyophilized. After thorough mixing, the mixture was applied only on the surface of a carbon paste electrode which was already filled with approximately 40 mg of carbon paste, and polished on a filter paper.

This electrode (enzyme electrode) was treated in a 10 mM MOPS buffer solution (pH7.0) containing 1% glutaraldehyde for 30 minutes at room temperature, and further treated in a 10 mM Tris buffer solution (pH7.0) for 20 minutes at room temperature. This electrode was equilibrated in a 10 mM MOPS buffer solution (pH7.0) for one hour or more at room temperature.

### Example 5

### Measurement of Glucose Using a Sensor Constructed from PQQGDH, Cytochrome b562 and Potassium Ferricyanide as an Electron Mediator

A 10 mM MOPS buffer solution (pH7.0) containing 1 mM CaCl₂ was placed in a constant temperature cell, potassium ferricyanide was added as a mediator at a final concentration of 10 mM, and the total volume was made to be 10 ml. The carbon paste electrode (enzyme electrode) constructed in Example 4, in which PQQGDH and cyt.b562 are immobilized, as the working electrode, a platinum electrode as the counter electrode and an Ag/AgCl electrode as the reference electrode were inserted therein to construct the sensor.

Measurements were all performed at 25° C. An electric potential of +400 mV vs Ag/AgCl was applied. When the current became stationary, the current value that increased with the addition of different concentrations of glucose was measured. The current value when glucose was not added was defined as 0 A.

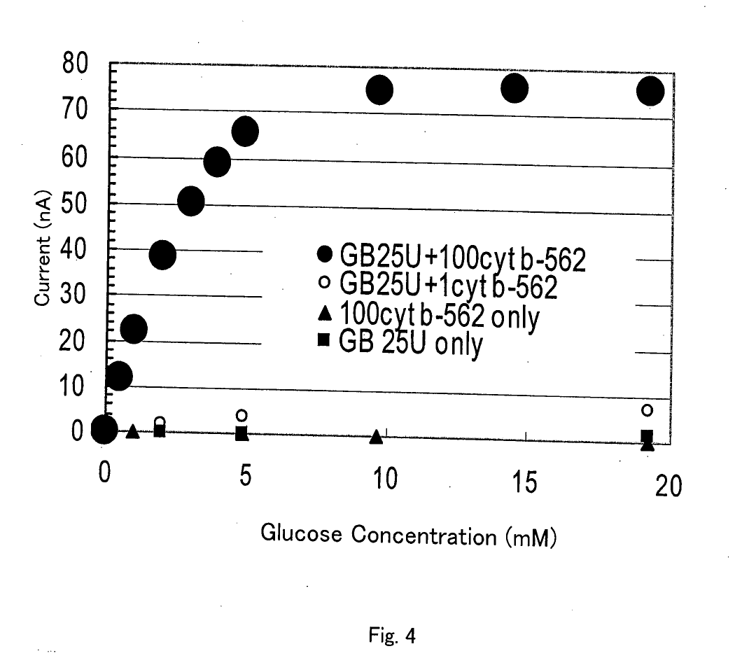

The enzyme electrodes used were an enzyme electrode in which PQQGDH alone is immobilized, an enzyme electrode in which equal molar of cyt.b562 to PQQGDH are immobilized, an enzyme electrode in which 100 times molar excess of cyt.b562 to PQQGDH is immobilized, and an enzyme electrode in which cyt.c is immobilized but not containing PQQGDH. The dependencies to glucose concentration of each response current value are shown in FIG. 4. The response current values of respective electrode at the glucose concentration of 5.0 mM were compared. The response current values of each electrode at the glucose concentration of 5.0 mM were less than 1.0 nA for the enzyme electrode in which PQQGDH alone is immobilized, 0 nA for the enzyme electrode in which cyt.b562 alone is immobilized, 5 nA for the enzyme electrode in which equal molar of cyt.b562 to PQQGDH is immobilized and 65 nA for the enzyme electrode in which 100 times molar excess of cyt.b562 to PQQGDH is immobilized. The current value of the electrode with immobilized cyt.b562 in response to glucose was approximately 60 times higher compared to the electrode with immobilized PQQGDH alone.

### Example 6

### Construction of an Enzyme Electrode in which PQQGDH and Cytochrome b562 are Immobilized

PQQGDH (25 units, 0.64×10⁻¹⁰ mol) and cyt.b562 sample (100 times molar excess to the enzyme, i.e., 0.64×10⁻⁸ mol) prepared in the same way as in Example 4 were mixed together with 20 mg of carbon paste and lyophilized. After thorough mixing, the mixture was applied only on the surface of a carbon paste electrode which was already filled with approximately 40 mg of carbon paste, and polished on a filter paper.

This electrode (enzyme electrode) was treated in a 10 mM MOPS buffer solution (pH7.0) containing 1% glutaraldehyde for 30 minutes at room temperature, and further treated in a 10 mM Tris buffer solution (pH7.0) for 20 minutes at room temperature. This electrode (enzyme electrode) was equilibrated in a 10 mM MOPS buffer solution (pH7.0) for one hour or more at room temperature.

### Example 7

### Measurement of Glucose Using an Enzyme Electrode in Which PQQGDH and Cytochrome b562 are Immobilized

A 10 mM MOPS buffer solution (pH7.0) containing 1 mM CaCl₂ was placed in a constant temperature cell and the total volume was made to be 10 ml without adding a mediator. The carbon paste electrode (enzyme electrode) constructed in Example 6, in which PQQGDH and cyt.b562 are immobilized, as the working electrode, a platinum electrode as the counter electrode and an Ag/AgCl electrode as the reference electrode were inserted therein to construct the sensor.

Measurements were all performed at 25° C. An electric potential of +400 mV vs Ag/AgCl was applied. When the current became stationary, the current value that increased with the addition of different concentrations of glucose was measured. The current value when glucose was not added was defined as 0 A.

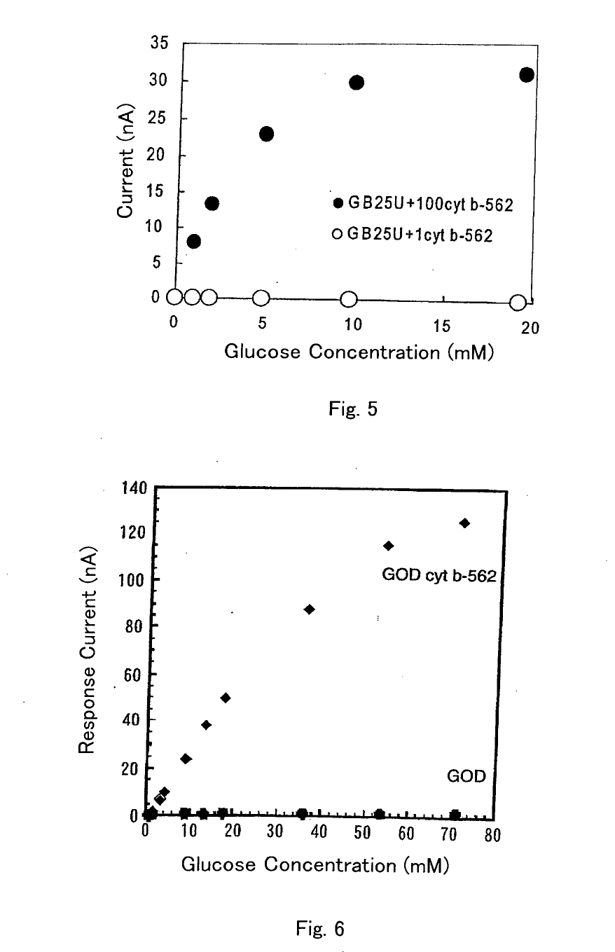

FIG. 5 shows the calibration curves of the enzyme electrode in which equal molar of cyt.b562 to PQQGDH is immobilized and the enzyme electrode in which 100 times molar excess of cyt.b562 to PQQGDH is immobilized. The response current values of respective electrodes at a glucose concentration of 5.0 mM were compared. The response current values of each electrode obtained at a glucose concentration of 10 mM were 0 nA for the enzyme electrode in which equal molar of cyt.b562 to PQQGDH is immobilized and 30 nA for the enzyme electrode in which 100 times molar excess of cyt.b562 to PQQGDH is immobilized. Thus, it is shown that by using an electrode in which PQQGDH and cyt.b562 are immobilized, glucose can be measured even in a condition where an electron mediator is not added to the sensor system.

### Example 8

### Sensor Comprising an Enzyme Electrode in Which Glucose Oxidase (GOD) and Recombinant cyt.b562 are Immobilized and a Mediator

Five units of Aspergillus niger-derived glucose oxidase (101 U/mg protein), 4.3×10⁻⁸ mol (corresponds to 100 times molar excess to GOD, i.e., 0.6 mg) of Cytb562 produced by recombinant E. coli and 20 mg of carbon paste were mixed, lyophilized, and applied to a carbon paste electrode. This electrode was immersed in a 1% glutaraldehyde aqueous solution for 30 minutes to crosslink the proteins with each other. The enzyme electrode constructed in this way was used as the working electrode, Ag/AgCl was used as the reference electrode and Pt electrode was used as the counter electrode. The electrodes were inserted in a 10 mM potassium phosphate buffer solution (pH7.0) containing 10 mM potassium ferricyanide as a mediator, and the response current value upon addition of cholesterol was measured at 25° C. in a batch system. The applied electric potential was +400 mV vs Ag/AgCl. As a control, a GOD-immobilized electrode that does not contain Cytb562 was constructed in the same manner, and the response to the addition of glucose was measured.

The result is shown in FIG. 6. Even in the case of the control electrode without Cytb562, an increase in the response current value to glucose is observed. However, in this measurement system, the response current value is as low as 3 nA at 10 mM glucose. In contrast, the GOD electrode with Cytb562 showed a satisfactory responsiveness. It showed a response of higher than 60 nA at 10 mM glucose, which is more than 20 times greater than the electrode without Cytb562.

### Example 9

### Direct Electron Transfer Type Sensor Comprising an Enzyme Electrode in Which Glucose Oxidase (GOD) and cyt.b562 are Immobilized

Five units of Aspergillus niger-derived glucose oxidase (69 U/mg protein), 4.3×10⁻⁸ mol (corresponds to 100 times molar excess to GOD, i.e., 0.6 mg) of Cytb562 produced by recombinant E. coli and 20 mg of carbon paste were mixed, lyophilized, and applied to a carbon paste electrode. This electrode was immersed in a 1% glutaraldehyde aqueous solution for 30 minutes to crosslink the proteins with each others. The enzyme electrode constructed in this way was used as the working electrode, Ag/AgCl was used as the reference electrode and Pt electrode was used as the counter electrode. The electrodes were inserted in a 10 mM potassium phosphate buffer solution (pH7.0) containing 10 mM potassium ferricyanide, and a cyclic voltammogram (CV) was measured at 25° C. Sweep rate was set to 50 mV/sec, and the electric potential was swept in a range of −300 mV to +300 mV. The change in the CV upon addition of 20 mM glucose was measured in a batch system.

From the result of this experiment, the current value in the vicinity of electric potential +300 mV (vs Ag/AgCl) clearly increased by the addition of glucose. Such a response is not observed with the electrode in which GOD alone is immobilized. From this fact, it is clear that a direct electron transfer type sensor without using an artificial electron mediator can be constructed using an enzyme electrode in which Cytb562 is immobilized together with GOD.

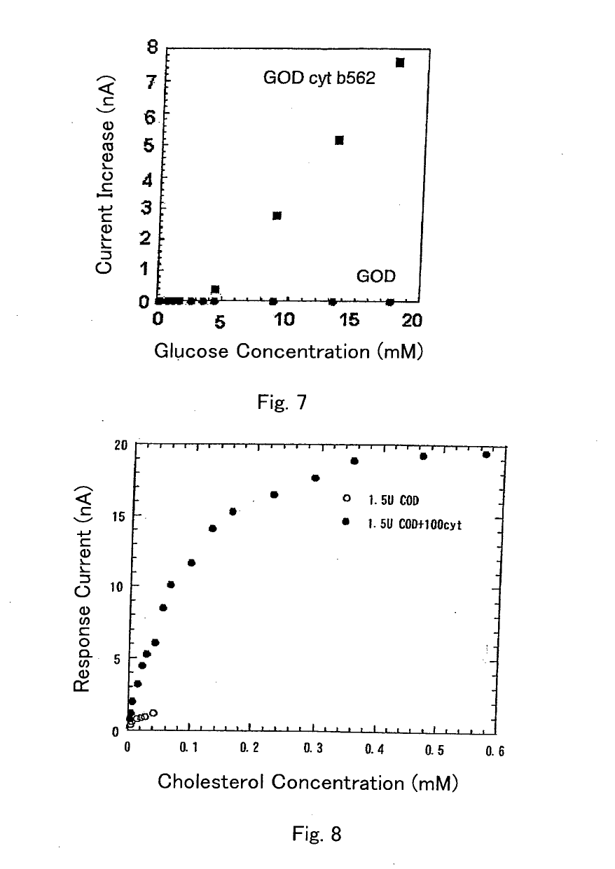

FIG. 7 shows the calibration curve of this electrode for glucose when the electric potential was fixed to +250 mV (vs Ag/AgCl). The response current values of each electrode at a glucose concentration of 5.0 mM were compared. No response current value was observed for the electrode in which GOD is immobilized alone, while in the electrode in which GOD was immobilized together with b562, response current values that depended on the glucose concentration were observed. Thus, it is shown that by using an electrode in which GOD and cyt.b562 are immobilized, glucose can be measured even in a condition where an electron mediator is not added to the sensor system.

### Example 10

### Sensor Comprising an Enzyme Electrode in Which Cholesterol Oxidase (COD) and cyt.b562 are Immobilized and a Mediator

One and a half units (5.26×10⁻¹⁰ mol) of cholesterol oxidase (COD; 12.77 U/mg protein), 5.26×10⁻⁸ mol (corresponds to 100 times molar excess to COD, i.e., 0.789 mg) of Cytb562 produced by recombinant E. coli and 20 mg of carbon paste were mixed, lyophilized, and applied to a carbon paste electrode. This electrode was immersed in a 1% glutaraldehyde aqueous solution for 30 minutes to crosslink the proteins with each other. The enzyme electrode constructed in this way was used as the working electrode, Ag/AgCl was used as the reference electrode and Pt electrode was used as the counter electrode. The electrodes were inserted in a 10 mM potassium phosphate buffer solution (pH7.0) containing 10 mM potassium ferricyanide as a mediator, and the response current value upon addition of cholesterol was measured at 25° C. in a batch system. The applied electric potential was +400 mV vs Ag/AgCl. As a control, a COD-immobilized electrode that does not contain Cytb562 was constructed in the same manner, and the response to the addition of cholesterol was measured. The cholesterol solution was prepared by mixing 5.0 mg of Triton X-100 and 500 mg of cholesterol and heat-melting, 90 ml of distilled water was added, boiled and cooled, then 4.0 g of sodium cholate salt was added and dissolved, then distilled water was added to obtain a total volume of 100 ml, which served as the standard solution.

The result is shown in FIG. 8. Even in the case of the control electrode without Cytb562, an increase in the response current value to cholesterol was observed. However, in this measurements system, the response current value was as low as 1 nA at a cholesterol concentration of 0.02 mM. At higher concentrations the response was nearly saturated. In contrast, the COD electrode with Cytb562 showed a satisfactory responsiveness. It showed a response of higher than 2 nA in 0.02 mM cholesterol, which is 20 times greater than the electrode without Cytb562. In addition, the response did not saturate for high concentrations of cholesterol, such that measurements of concentrations above 0.5 mM was also effected. The response current value at that concentration was higher than 20 nA, which was nearly 20 times the maximum value of response value for the electrode without Cytb562.

### Example 11

### Sensor Comprising an Enzyme Electrode in Which Fructosylamine Oxidase (FAOD) and Cytochrome b562 are Immobilized and a Mediator

Pichia sp. N1-1 strain-derived fructosylamine oxidase (JP A 2000-270855) was used. Fructosylamine oxidase was dissolved in a 10 mM potassium phosphate buffer solution (pH7.0), and was dialyzed overnight against 10 mM potassium phosphate buffer solution (pH7.0). The measurement of FAOD activity was performed by adding 20 μl of 15 mM 4-amino-antipyrine, 20 mM phenol, 20 U/ml peroxidase and 1 M fructosyl valine at 25° C., and measuring the change in the optical density at 500 nm using a spectrophotometer. The enzymatic activity that generates 1 pmol H₂O₂ in 1 minute was defined as 1 U, and the molar extinction coefficient was defined as 12880 mM⁻¹.

Enzyme electrodes in which FAOD is immobilized was constructed as in Example 2, using FAOD only (0.4 units, 5.08×10⁻⁹ mol), FAOD and 20 times molar excess of cytb₅₆₂ (1.01×10⁻⁷ mol), or FAOD and BSA (the same amount of protein as above). A 10 mM MOPS buffer solution (pH7.0) containing 1 mM CaCl₂ was placed in a constant temperature cell, potassium ferricyanide was added as a mediator at a final concentration of 10 mM, the total volume was made to be 10 ml, then argon gas was blown. The carbon paste electrode was used as the working electrode, in combination with a platinum electrode as the counter electrode and an Ag/AgCl electrode as the reference electrode. Measurements were all performed at 25° C., with an applied voltage of +100 mV vs Ag/AgCl. When the current became stationary, the current value that increased with the addition of various concentrations of fructosyl-valine solutions was measured. The current value when fructosyl-valine was not added was defined as 0 A.

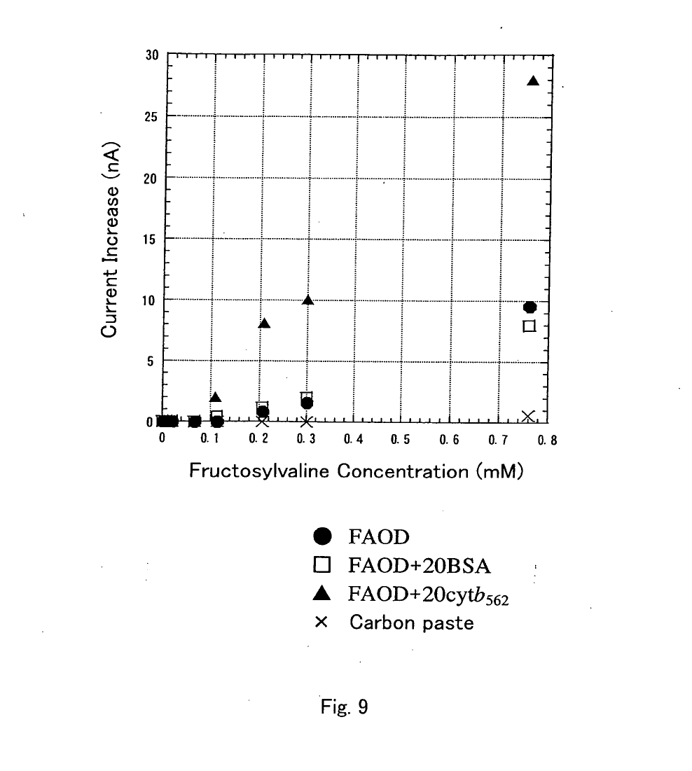

The calibration curve of each electrode is shown in FIG. 9. In the case where FAOD was immobilized together with cytb₅₆₂, the current value in response to fructosyl-valine was 4 times higher compared to the electrodes of FAOD alone or FAOD+100BSA. In addition, in the system that does not contain m-PMS, a response to fructosyl-valine could not be obtained for any of the electrodes.

### Example 12

### Database Search of Proteins Similar to cytb₅₆₂

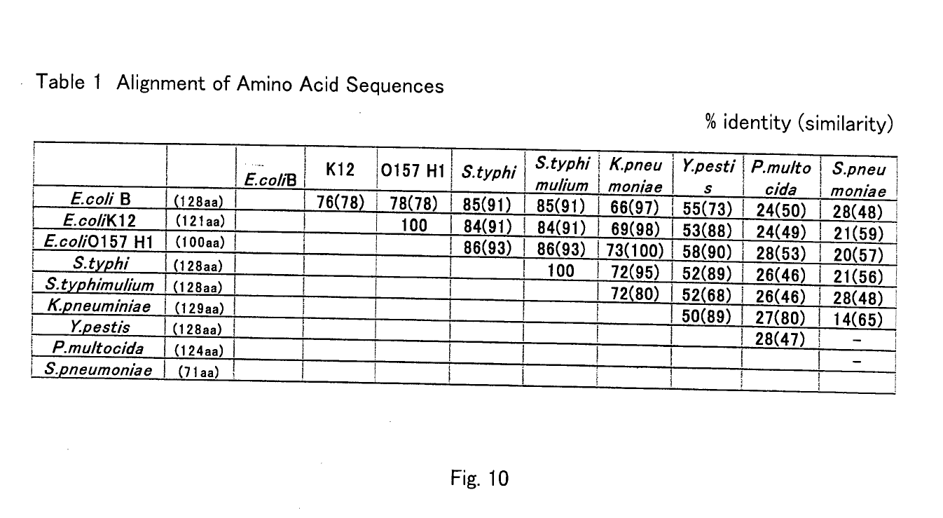

A homology search was performed for E. coli B-derived water-soluble cytb₅₆₂ against amino acid sequences derived from various living organisms for which the genomic information is published (FIG. 10). Sequences that are highly homologous to water-soluble cytb₅₆₂ were found in Salmonella typhi CL18 and Yesinia pestis C092, in addition to other E. coli strains. Y. pestis is a gram-negative bacterium and is a bacterial pathogen of pest. A homologous sequence was also found in Pasteurella multocida PM70, a gram-negative short bacillus known to be the pathogen for infectious diseases such as haemorrhagic septicaemia in livestock. A low similarity region was identified in residues 210 to 280 among the 619 residues of PskpA protein from Streptococcus pneumoniae R6. Pneumococcal surface protein A (PspA) is thought to be a protein for child immunity to S. pneumoniae, which is responsible for infectious diseases such as encephalitis, and similarity is present in the a-helix region of the first half of the 619 residues.

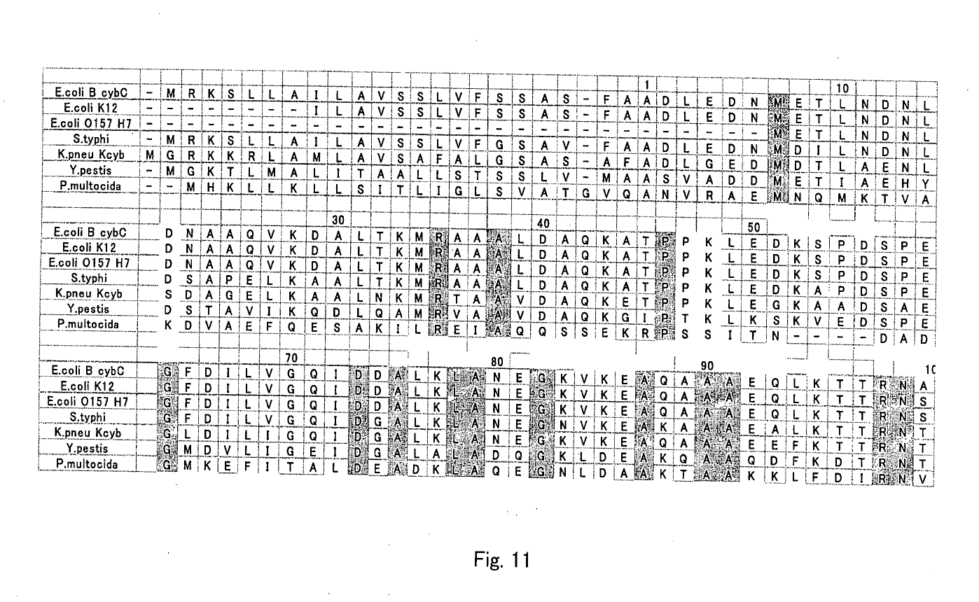

In addition, based on the results of these amino acid homology searches, the residues conserved among the polypeptides were searched. It is shown that Met7 on the N-terminal side and His102 on the C-terminal side, which coordinate the haeme iron, are conserved, and as far as the entire sequence, the C-terminal region is relatively conserved (FIG. 11). Based on this result, when the residues that are conserved were represented spatially on the three-dimensional structure of cytb₅₆₂, it was found that several conserved amino acid residues exist on the 4^{th} a-helix, and that some residues with an aromatic ring are conserved, including the Phe residues at the 61^{st} and the 65^{th} positions (numbers exclude the signal sequence) and the Tyr residue at the 105^{th} position, which are in the vicinity of the haeme iron, and the His residue at the 102^{nd} position, which coordinates the haeme iron. This result is consistent with the report that aromatic amino acids (Phe82, Trp86 and Phe125) positioned in the vicinity of the haeme are conserved in cytc′, whose structure is similar to cytb₅₆₂ (PC Weber, FR Salemme (1981) J. Biol. Chem. 256, 7702-7704). In the same way as cytb₅₆₂, a 4-a-helix bundle is formed in cytc′, and the similarity in amino acid residues in the region with the same structure is as low as 17%. However, from the structure analysis of both proteins, it is shown that the three-dimensional positions of His102 (cytb₅₆₂) and His122 (cytc′), which coordinate the haeme iron of cytc′ and cytb₅₆₂, are matching and that there is similarity in the arrangement of the aromatic amino acid residues present at equivalent positions in the vicinity of the haeme (cytb₅₆₂: Phe61, Phe65 and Tyr105) (cytc′: Phe82, Trp86 and Phe125) and the haeme (PC Weber, FR Salemme (1981) J. Biol. Chem. Hamada K, PH Bethget and FS Mathews (1995) J. Mol. Biol. PD Barker and AR Fersht (1999) Biochemistry 38, 8657-8670). The cytb₅₆₂ haeme is positioned in an internal pocket of hydrophobic residues, and is coordinated by His102 and Met7 in a state where the proto-haeme and its propionic acid side-chain are exposed to solvent. In this arrangement, the side chain of Phe65 present in the vicinity of the haeme is positioned in parallel to the proto haeme, and forms a hydrogen bond. From the fact that Phe61 and Tyr105 are also interacting with the proto-haeme, it is thought that these residues are important for the orientation of the haeme. In addition, the spatial positions of His102 (cytc′:122) and Met7 (cytc′:16 not coordinated), which coordinate the haeme iron, are determined by the positions of the 1^{st} and 4^{th} helices of cyt. In particular, the shape of the 4^{th} a-helix of the holo-type and the apo-type show no changes. Also the spatial positions of Cys118 and Cys121 on the 4^{th} helix, which make thiol bonds to the haeme of cytc′, correspond to the spatial positions of Arg98 and Tyr101 of cytb₅₆₂. Taking together, it is thought that the structure of the 4^{th} a-helix is important in the protein-haeme interaction. From the result of the alignment shown in FIG. 11, this is consistent with the fact that there was a good conservation with respect to the 4^{th} helix. It is thought that the conservation of the aromatic residues and the residues on the a-helices, which are thought to strengthen the interaction between such characteristic structure called 4-α-helix bundle and the haeme, allows the haeme to adopt a specific orientation and a common arrangement. It is believed that haeme is subject to the influence of solvent (pH) because it is highly exposed to the solvent.

### Example 13

### Preparation of K. Pneumoniae-Derived Cytochrome C b562

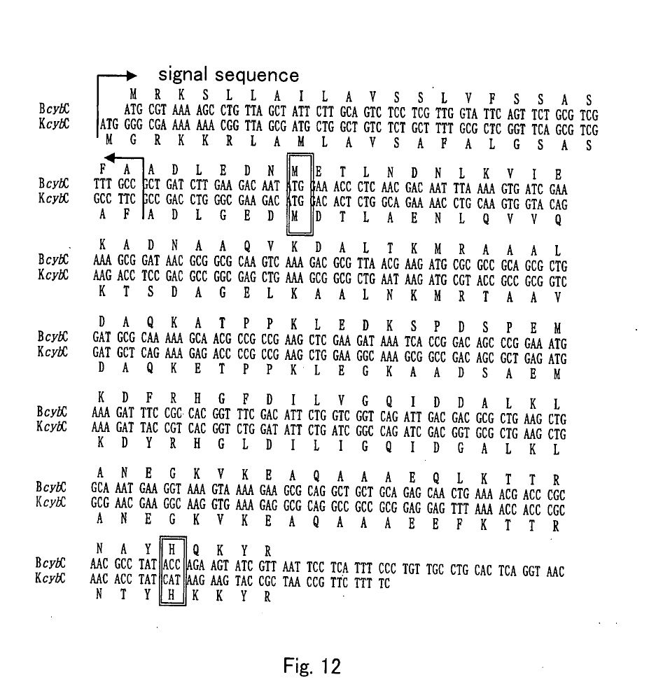

Based on the published genome information on Klebsiella pneumoniae MGH78578, BLAST was used to conduct a homology search with the amino acid sequence and the nucleotide sequence of water-soluble cytochrome (cybc) b₅₆₂ from Escherichia coli B, and a region with a high similarity was identified. Primers which flank this region and have restriction endonuclease sites (NcoI/BamHI) were designed. PCR amplification was performed on K. pneumoniae NCTC418 genome using these primers, and an amplification fragment of approximately 400 bp was obtained. When the nucleotide sequence was compared to the cybc gene, it had a similarity of 70% at the nucleotide level, and 67% at the amino acid level (FIG. 12).

The NcoI-BamHI fragment of this PCR product was subcloned into the expression vector pTrc99A described in Example 1 and used for transformation of Escherichia coli DH5a strain. Escherichia coli that contain the gene coding for Klebsiella pneumoniae-derived cytochrome Cb562 (KNcyt.b) were cultured and red cells were obtained. The spectra of the periplasmic, water-soluble and membrane fractions of these cells showed peaks that are characteristic of the oxidized form (418 nm) and the reduced form (428 nm, 562 nm) of cytochrome in both the periplasmic fraction and the water-soluble fraction.

Purification of KNcyt.b was performed as indicated below. Escherichia coli DH5a that contains the gene coding for KNcyt.b was cultured in 7 L scale in LB medium at 37° C. at 200 rpm, and the cells were collected at 7,000×g for 5 min at 4° C., washed with 50 mM p.p.b. (pH7.0) and frozen overnight at −80° C. These cells were suspended and lysed in 50 mM p.p.b. (pH7.0), and centrifuged (10,000×g, 20 min, 4° C.). HCl was added to the supernatant to adjust to pH4-5, stirred for 1 hour at 4° C., and NaOH was added to adjust to pH7. Ultracentrifugation (50,000 rpm, 60 min, 4° C.) was performed and the resulting supernatant was dialyzed overnight against a 10 mM MOPS buffer solution (pH7.2). To this sample, potassium ferricyanide (10 mM final concentration) was added to oxidize Cyt b₅₆₂, and desalted with PD-10. This sample was subjected to an anion exchange column chromatography (DEAE-5PW, A: 10 mM MOPS pH7.2, B: 300 mM NaCl, 10 mM MOPS pH7.2, 80% gradient, 9 column volumes) and gel filtration (Superdex200, 300 mM NaCl, 10 mM MOPS pH7.2) to obtain purified cytb₅₆₂, which was concentrated using PEG. A single band of about 14 kDa was observed in SDS-PAGE.

The concentration of KNcyt. b was determined as indicated below. The spectrum of the oxidized form between 300 nm and 600 nm was measured and the peaks that are characteristic of the oxidized form (418 nm, 533 nm) were identified, then a reducing agent (sodium hydrosulfite) was added, and peaks that are characteristic of the reduced form (427 nm, 531 nm, 562 nm) were measured. Difference in the optical density of the reduced form <ABS₅₆₂ nm-ABS₅₇₈ nm>was determined, and the concentration was calculated using the molar extinction coefficient of E. coli B-derived cytb₅₆₂ according to the calculation equation:
KNcyt.b concentration(mM)=ABS_{562 nm-578 nm}×24.6× dilution factor

### Example 14

### Oxido-Reduction Potential of the K. Pneumoniae-Derived Cytochrome C b562

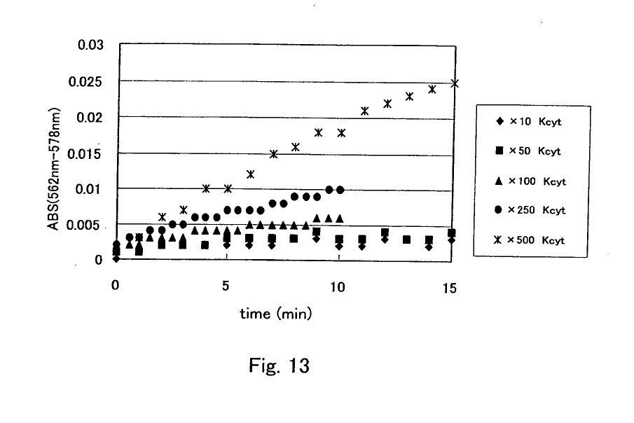

PQQ and CaCl₂ (final concentrations of 1 μM and 1 mM, respectively) were added to PQQGDH-B at room temperature for 30 minute to convert it into the holo form. The sample was dialyzed overnight (10 mM MOPS pH7.0, 1 mM CaCl₂) to remove excess PQQ. To the enzyme sample, different amounts of KNcyt.b and 0.5 U of GDH-B were added, then glucose (50 mM final concentration) was added and the increase in the reduced form cytb per unit time was determined based on the difference spectrum between 562 nm and 578 nm. An increase in the reduced peak was observed by the addition of glucose under the presence of PQQGDH-B. In addition, a concentration dependency was observed for the increase in the ratio of the reduced form of KNcyt.b to the concentration of PQQGDH-B (mol/l) (FIG. 13), which indicates that a direct electron transfer occurred between KNcyt.b and PQQGDH-B.

### Example 15

### Enzyme Electrode in Which PQQGDH and K. Pneumoniae-Derived Cytochrome Cb562 are Immobilized and Measurement of Glucose

PQQGDH (25 units, 0.64×10⁻¹⁰ mol) and KNcyt.b562 sample (100 times molar excess to the enzyme, i.e., 0.64×10⁻⁸ mol) prepared in Example 13 were used to create an enzyme electrode in the same way as in Example 2.

A 10 mM MOPS buffer solution (pH7.0) containing 1 mM CaCl₂ was placed in a constant temperature cell, potassium ferricyanide was added as a mediator at a final concentration of 10 mM, and the total volume was made to be 10 ml. The carbon paste electrode (enzyme electrode), in which PQQGDH and KNcyt.b562 are immobilized, as the working electrode, a platinum electrode as the counter electrode and an Ag/AgCl electrode as the reference electrode were inserted in the cell to construct the sensor. The measurement was performed in the same way as in Example 3. The electrode in which PQQGDH and KNcyt.b are immobilized showed a significantly higher response current value compared to the electrode in which PQQGDH alone is immobilized.

Industrial Utility

The enzyme electrode of the present invention and biosensor using the electrode are useful as glucose sensors for measuring blood glucose levels, and as sensors for measuring the concentrations of cholesterol and fructosylamine in the blood.

1. An enzyme electrode having an oxidoreductase and an electron-transfer protein.

2. The enzyme electrode of claim 1, wherein the oxidoreductase is an oxidoreductase having pyrroloquinoline quinone as coenzyme.

3. The enzyme electrode of claim 1, wherein the oxidoreductase is an enzyme having flavin as coenzyme.

4. The enzyme electrode of claim 1, wherein the oxidoreductase is selected from the group consisting of glucose oxidase, cholesterol oxidase, lactate oxidase, alcohol oxidase, galactose oxidase, bilirubin oxidase, fructosylamine oxidase, glucose dehydrogenase, alcohol dehydrogenase and glucose-3-dehydrogenase.

5. The enzyme electrode of claim 1, wherein the electron-transfer protein is cytochrome C.

6. The enzyme electrode of claim 1, wherein the electron-transfer protein is cytochrome b562.

7. The enzyme electrode of claim 1, wherein the electron-transfer protein is a protein having the amino acid sequence from Ala24 to Arg129 of SEQ ID NO: 6 or from Ala24 to Arg129 of SEQ ID NO: 8.

8. The enzyme electrode of claim 1, wherein the electron-transfer protein is cytochrome c551.

9. The enzyme electrode of claim 1, wherein the oxidoreductase is glucose dehydrogenase and the electron-transfer protein is cytochrome b562.

10. The enzyme electrode of claim 1, wherein the oxidoreductase is cholesterol oxidase and the electron-transfer protein is cytochrome b562.

11. The enzyme electrode of claim 1, wherein the oxidoreductase is lactate oxidase and the electron-transfer protein is cytochrome b562.

12. The enzyme electrode of claim 1, wherein the oxidoreductase is fructosylamine oxidase and the electron-transfer protein is cytochrome b562.

13. The enzyme electrode of claim 1, wherein the oxidoreductase is glucose dehydrogenase and the electron-transfer protein is cytochrome b562.

14. The enzyme electrode of claim 1, wherein the oxidoreductase is glucose dehydrogenase having pyrroloquinoline quinone as coenzyme (PQQGDH) and the electron-transfer protein is cytochrome b562.

15. The enzyme electrode of claim 1, wherein the oxidoreductase is glucose dehydrogenase having flavin as coenzyme and the electron-transfer protein is cytochrome b562.

16. An enzyme electrode characterized in that glucose dehydrogenase and cytochrome C are attached onto an electrode in a state wherein they are chemically crosslinked.

17. An enzyme electrode characterized in that glucose dehydrogenase and cytochrome b562 are attached onto an electrode in a state where they are chemically crosslinked.

18. The enzyme electrode of claim 16 or 17, wherein crosslinking is effected using glutaraldehyde.

19. The enzyme electrode of claim 6, wherein cytochrome b562 is Escherichia coli-derived cytochrome b562.

20. A sensor characterized in that it uses the enzyme electrode of any of claim 1 as working electrode.

21. The sensor of claim 20 further containing an electron mediator.

22. The sensor of claim 21, wherein the electron mediator is selected from potassium ferricyanide, phenazine methosulfate, ferrocene and derivatives thereof.

23. The sensor of claim 21, wherein PQQGDH and cytochrome C are attached onto an electrode in a state where they are chemically crosslinked, and wherein the electron mediator is potassium ferricyanide.

24. The sensor of claim 21, wherein PQQGDH and Escherichia coli cytochrome b562 are attached onto an electrode in a state where they are chemically crosslinked, and wherein the electron mediator is potassium ferricyanide.
